(19) United States
(12) Patent Application Publication
(10) Pub. No.: US 2021/0383910 A1
(43) Pub. Date: Dec. 9, 2021

(54) SYSTEM AND METHOD FOR AUTOMATICALLY AND CONTINUOUSLY MONITORING MEDICATION DELIVERY TO A PATIENT

(71) Applicant: The Methodist Hospital, Houston, TX (US)

(72) Inventor: Brian PARRISH, Houston, TX (US)

(21) Appl. No.: 17/290,823

(22) PCT Filed: Oct. 31, 2019
(86) PCT No.: PCT/US19/59143

§ 371 Date: May 3, 2021

Related U.S. Application Data
(60) Provisional application No. 62/754,031, filed on Nov. 1, 2018.

(51) Int. Cl. G16H 20/13; G16H 10/60; G16H 70/40

(52) U.S. Cl. CPC G16H 20/13; G16H 70/40; G16H 10/60

(57) ABSTRACT

A system for automatically monitoring the delivery of medication to a patient, the system comprising: an electronic medical record containing information regarding the medical condition of the patient; a prescription order identifying a medication to be delivered to the patient; a database identifying contraindications and warnings for specific medications; a rules engine for determining the appropriateness of a medication vis-a-vis a medical condition of a patient; and a central processing unit (CPU) configured to (i) provide the prescription order and the electronic medical record of the patient to the rules engine, (ii) receive a determination from the rules engine regarding the appropriateness of the prescription order vis-a-vis the medical condition of the patient, and (iii) mark the electronic medical record with an indication regarding the appropriateness of the prescription order vis-a-vis the medical condition of the patient.

### REFERENCE TO PENDING PRIOR PATENT APPLICATION

This patent application claims benefit of pending prior U.S. Provisional Patent Application Ser. No. 62/754,031, filed Nov. 1, 2018 by The Methodist Hospital and Brian Parrish for SYSTEM AND METHOD FOR AUTOMATICALLY MONITORING MEDICATION DELIVERY TO A PATIENT IN ORDER TO REDUCE MEDICATION DELIVERY ERRORS (Attorney's Docket No. METHODIST-26A PROV), which patent application is hereby incorporated herein by reference.

### FIELD OF THE INVENTION

This invention relates generally to systems and methods for monitoring medication delivery to a patient, and more particularly to systems and methods for automatically and continuously monitoring medication delivery to a patient in order to reduce medication delivery errors.

### BACKGROUND OF THE INVENTION

Deaths and injuries resulting from medication delivery errors is a substantial burden on healthcare systems in general, and is particularly difficult for the individual patients involved. A medication delivery error is any preventable event that may cause or lead to inappropriate medication use or patient harm while the medication is in the control of a healthcare professional, patient or consumer. The sources of medication delivery errors include the administration of an incorrect drug, the administration of an incorrect concentration of a drug, the inappropriate administration of a drug, omitting the administration of a drug, etc. Furthermore, even with proper prescription orders, proper medication labeling and proper monitoring by the pharmacist dispensing the medications, medications may still be administered incorrectly due to the time delay which can occur between the time that a doctor prescribes a medication and the time that the medication is actually administered to the patient, e.g., where the patient's condition changes between the time that the medication is prescribed and the time that the medication is administered.

Currently, a prescription decision is made by a doctor based on the medical information which is available to the doctor at the time that the doctor prescribes the medication, and this prescription decision is thereafter reviewed by the pharmacist and then the prescription is placed into a patient's electronic medical record (EMR) so that one of the medical staff (typically a nurse) can thereafter administer the prescribed drug to the patient at the time(s) prescribed by the doctor. However, a patient's medical condition may change between (i) the time that the medication was prescribed by the doctor and reviewed by the pharmacist, and (ii) the time that the medication is actually delivered to the patient by a member of the medical staff (e.g., a nurse). The patient's medical condition may also change in the time between subsequent doses of the medication. In some cases this change in medical condition may render delivery of the prescribed medication no longer appropriate. Stated another way, in some cases, the doctor prescribes a medication that is appropriate to treat a patient's medical condition at the time that the medication is prescribed, but the patient's medical condition subsequently changes such that the prescribed medication is no longer appropriate.

For practical purposes, with current practices, the only way that there is an intervention in the scheduled delivery of a medication is if a member of the medical staff (e.g., a nurse) recognizes, prior to administering the medication, that the prescription is not appropriate (or is no longer appropriate) and raises the issue before administering the medication. Thus, an intervention by a member of the medical staff (e.g., a nurse) typically relies on: (i) the individual knowledge of the staff member involved (e.g., a nurse), and (ii) the comfort of the staff member (e.g., a nurse) to question the current appropriateness of the prescription. It will be appreciated that because of these factors, appropriate intervention may not always occur.

Thus there is a need for a new and improved system and method for automatically and continuously monitoring medication delivery to a patient in order to reduce medication delivery errors.

### SUMMARY OF THE INVENTION

The present invention comprises the provision and use of a new and improved system and method for automatically and continuously monitoring medication delivery to a patient in order to reduce medication delivery errors.

More particularly, the present invention comprises the provision and use of an automated monitoring system which continuously compares the known medication contraindications, and/or the known medication warnings and precautions, for each medication prescribed to a patient against the current clinical status of the patient. The automated monitoring system does this continuously and in “real-time”, prior to the delivery of the medication to the patient, and assigns an appropriate flag (e.g., a green “proceed” flag, a yellow “caution” flag or a red “stop” flag) to each medication delivery order, whereby to provide the medical staff with “real-time” information regarding the appropriateness of the medication delivery prior to that delivery. As a result, the automated monitoring system removes the burden of medication intervention decisions from the medical staff.

In one preferred form of the invention, there is provided a system for automatically monitoring the delivery of medication to a patient, the system comprising:

an electronic medical record containing information regarding the medical condition of the patient;

a prescription order identifying a medication to be delivered to the patient;

a database identifying contraindications and warnings for specific medications;

a rules engine for determining the appropriateness of a medication vis-à-vis a medical condition of a patient; and

a central processing unit (CPU) configured to (i) provide the prescription order and the electronic medical record of the patient to the rules engine, (ii) receive a determination from the rules engine regarding the appropriateness of the prescription order vis-à-vis the medical condition of the patient, and (iii) mark the electronic medical record with an indication regarding the appropriateness of the prescription order vis-à-vis the medical condition of the patient.

In another preferred form of the invention, there is provided a method for automatically monitoring the delivery of medication to a patient, the method comprising:

providing a system for automatically monitoring the delivery of medication to a patient, the system comprising:

an electronic medical record containing information regarding the medical condition of the patient;

a prescription order identifying a medication to be delivered to the patient;

a database identifying contraindications and warnings for specific medications;

a rules engine for determining the appropriateness of a medication vis-à-vis a medical condition of a patient; and

a central processing unit (CPU) configured to (i) provide the prescription order and the electronic medical record of the patient to the rules engine, (ii) receive a determination from the rules engine regarding the appropriateness of the prescription order vis-à-vis the medical condition of the patient, and (iii) mark the electronic medical record with an indication regarding the appropriateness of the prescription order vis-à-vis the medical condition of the patient; and

operating the central processing unit so as to (i) provide the prescription order and the electronic medical record of the patient to the rules engine, (ii) receive a determination from the rules engine regarding the appropriateness of the prescription order vis-à-vis the medical condition of the patient, and (iii) mark the electronic medical record with an indication regarding the appropriateness of the prescription order vis-à-vis the medical condition of the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a novel system for automatically monitoring medication delivery to a patient;

FIGS. 2 and 3 are schematic views showing exemplary details of the novel system shown in FIG. 1;

FIGS. 4-6 are schematic views showing how the novel system of FIGS. 1-3 may be used for reducing medication delivery errors to a patient;

FIG. 7 is a schematic view showing an exemplary network architecture which integrates the novel system of FIGS. 1-3 with an existing hospital network; and

FIG. 8 is a schematic view showing another exemplary network architecture which integrates the novel system of FIGS. 1-3 with an existing hospital network.

### DETAILED DESCRIPTION OF THE PREFERRED EMBODIMENTS

The present invention comprises the provision and use of a novel system for automatically and continuously monitoring medication delivery to a patient in order to reduce medication delivery errors.

### The Novel System in General

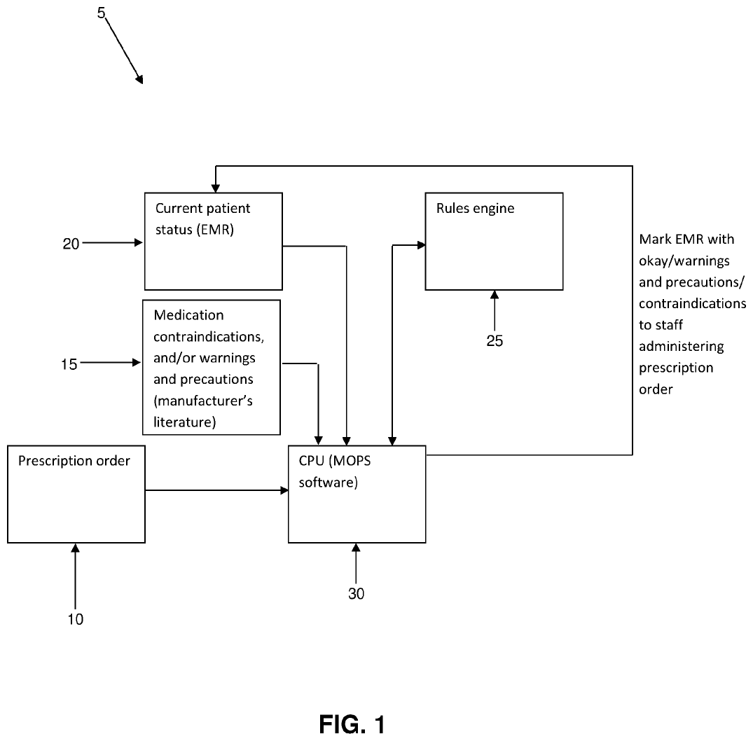

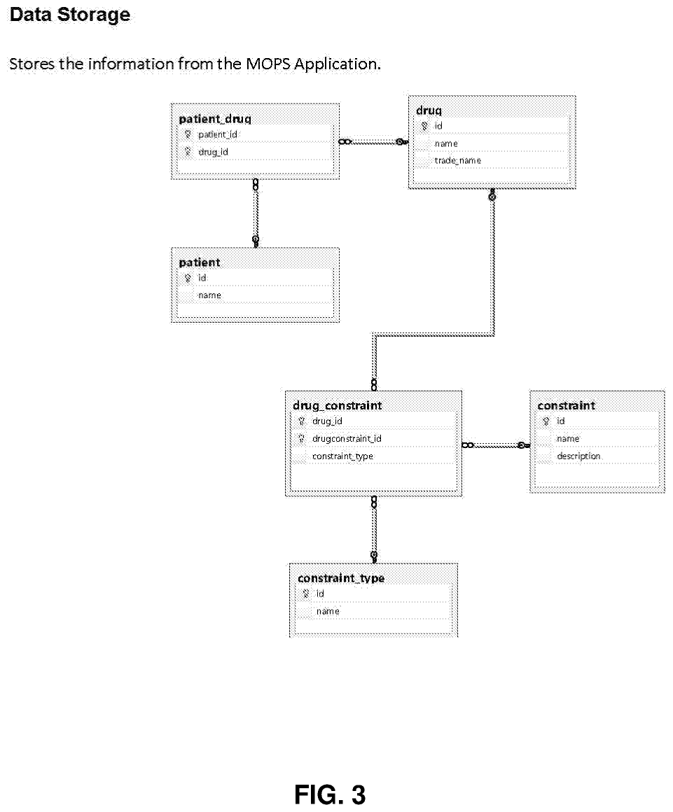

Looking first at FIGS. 1-3, there is shown a novel system 5 for automatically and continuously monitoring medication delivery to a patient in order to reduce medication delivery errors. System 5 generally comprises (i) a prescription input 10, (ii) a database 15, (iii) an electronic medical records (EMR) system 20, (iv) a rules engine 25, and (v) a central processing unit (CPU) 30 (sometimes also referred to herein as “the MOPS software” and/or “the MAPS software”).

Prescription input 10 generally comprises a prescription order, i.e., an order to deliver a particular medication to a particular patient in a particular dosage at a particular time, etc. The prescription order is generated by a doctor, nurse practitioner or other person authorized to prescribe medication. Prescription input 10 is communicated to CPU 30.

Database 15 generally comprises medication data which indicates medication contraindications, and/or medication warnings and precautions, based on medical databases (e.g., medical databases provided by third party vendors), medical literature (e.g., medical literature which is provided by drug manufacturers) or other data sources or databases. Database 15 is configured to communicate with CPU 30.

Database 15 may comprise a structured database or an unstructured database.

By way of example but not limitation, database 15 may comprise a structured database such as that provided by a third party vendor such as FDB® (First Databank), Medispan®, etc., or database 15 may comprise a structured database from another source, e.g., a healthcare provider database, a government database, etc. It will be appreciated that where database 15 comprises a structured database, database 15 also comprises appropriate tools for accessing and utilizing the data contained in the structured database.

By way of further example but not limitation, database 15 may comprise an unstructured database such as a compendium of IFUs (“Instructions For Use”) provided by a drug manufacturer, other literature provided by a drug manufacturer, scientific and medical literature, literature from a government source, etc. It will be appreciated that where database 15 comprises an unstructured database, database 15 also comprises appropriate tools for accessing and utilizing the data contained in the unstructured database, e.g., means for identifying medical contraindications, and/or medication warnings and precautions, associated with specific drugs and medical conditions.

EMR system 20 comprises a conventional electronic medical records system. EMR system 20 may comprise a commercially-available EMR system (e.g., Epic™, Cerner™, Allscripts™, etc.) or a “custom” EMR system. EMR system 20 stores data regarding the patient's current medical condition (e.g., physiologic measures such as weight, body temperature, heart rate, blood pressure, etc.; test results; diagnoses; progress notes; and/or other clinical information associated with a patient's medical condition), and is configured to communicate with CPU 30 so that CPU 30 can (i) receive patient status information from EMR system 20, and (ii) mark medication orders in EMR system 20 with an appropriate flag, as will be discussed in further detail below.

Rules engine 25 comprises a set of rules which assess the appropriateness of a prescription order vis-à-vis the medication contraindications, and/or the medication warnings and precautions, for a prescribed medication and the patient's current medical condition. Rules engine 25 communicates with CPU 30 so that it can receive (i) the prescribed medication (i.e., prescription input 10), (ii) the medication contraindications and/or the medication warnings and precautions, for the prescribed medication (i.e., from database 15), and (iii) the patient's current medical condition (i.e., from EMR system 20), and then send an assessment result back to CPU 30 after rules engine 25 analyzes the data from prescription input 10, database 15 and EMR system 20 according to the rules stored in rules engine 25.

CPU 30 generally comprises a general purpose computer and appropriate software for providing the functionality of the automated medication monitoring system of the present invention. CPU 30 receives the prescription order from prescription input 10 and determines the appropriateness of the prescription order in view of the patient's current medical status. This is performed by retrieving the medication contraindications (and/or the medication warnings and precautions) for the prescribed drug from database 15, retrieving the patient's current medical status from EMR system 20, and using rules engine 25 to assess the appropriateness of the prescribed drug vis-à-vis the patient's current medical status. CPU 30 then assigns an appropriate flag (e.g., a green “proceed” flag, a yellow “caution” flag or a red “stop” flag) next to each medication order in the patient's EMR system 20. In one form of the invention, the “flags” assigned by CPU 30 are colored flags (e.g., green flags, yellow flags, red flags, etc.). However, it should also be appreciated that, if desired, the “flags” may comprise particular shapes and/or symbols (e.g., a “checked box”, a triangle, an octagon, etc.) having associated colors (e.g., a green “checked box”, a yellow triangle, a red octagon, etc.). Furthermore, it should be appreciated that the term “flags” is intended to encompass substantially any visual and/or audible que which is deliverable through EMR system 20 to a user so as to indicate the appropriateness/inappropriateness of a prescription in view of the patient's current medical status.

In one preferred form of the invention, a red flag is assigned when medication contraindications are found for prescription input 10, a yellow flag is assigned when medication warnings and precautions are found for prescription input 10, and a green flag is assigned when no medication contraindications, and no medication warnings and precautions, are found for prescription input 10.

In another preferred form of the invention, when CPU 30 assigns a yellow “caution” flag (i.e., for when medication warning(s) and precaution(s) are indicated) or a red “stop” flag (i.e., for when medication contraindication(s) are indicated) next to a medication order, CPU 30 also provides explanatory information along with the yellow “caution” flag and red “stop” flag in order to explain the reason(s) for the assigned flag and present any information that needs to be communicated to the prescriber of the medication or the pharmacist (or to other medical staff, e.g., a nurse).

In one form of the present invention, a yellow “caution” flag (or a red “stop” flag) provides the medical staff (e.g., a nurse) with options for proceeding, and offers explanations based on established literature and/or a respected/peer reviewed clinical decision support tool (CDST). The explanation accompanying a yellow “caution” flag (or a red “stop” flag) preferably includes a hyperlink to the literature relied on for the explanation, so that the medical staff (e.g., a nurse) can access the literature that supports the explanation.

In addition, if desired, a yellow “caution” flag (or a red “stop” flag) may require acknowledgement from the medical staff (e.g., a nurse), and/or may prompt the medical staff to provide a reason for ignoring/overriding a yellow “caution” flag (or a red “stop” flag). Furthermore, if desired, a yellow “caution” flag (or a red “stop” flag) may require an override from a user having greater system privileges (e.g., a user with administrative level function privileges), and an override requirement may lead to suspension of further delivery of the medication if the required override is not received within a specified amount of time (e.g., 12 hours).

It should be appreciated that, where the prescription order calls for repeated doses of a medication, CPU 30 automatically and continuously monitors the patient's condition in EMR system 20, and automatically updates the flags next to each medication delivery in EMR system 20 upon each change in the patient's medical condition recorded in EMR system 20, so that up-to-date flags will be seen by the medical staff accessing EMR system 20. In other words, CPU 30 not only automatically checks the appropriateness of a prescription vis-à-vis the patient's medical condition at the time that the prescription order is entered via prescription input 10, but also automatically checks the appropriateness of the medication when updated medical status information is introduced into EMR system 20, so that the automated medication monitoring system tests the appropriateness of a medication delivery using the latest information in EMR system 20.

In general, the components of system 5 are configured to operate as follows:

(i) prescription input 10 is sent to CPU 30;

(ii) CPU 30 communicates with database 15 and EMR system 20 in order to gather information relating to prescription input 10 (i.e., medication contraindications, and/or medication warnings and precautions, from database 15 and to gather the current patient status from EMR system 20);

(iii) CPU 30 communicates the prescription order, the information received from database 15 (i.e., medication contraindications, and/or medication warnings and precautions), and the information received from EMR system 20 (i.e., current patient status) to rules engine 25;

(iv) rules engine 25 analyzes the data from CPU 30 (i.e., the prescribed drug, the medication contraindications and/or the medication warnings and precautions for the prescribed drug, and the current medical status of the patient) and sends a result (i.e., “proceed”, “caution”, or “stop”) back to CPU 30; and

(v) CPU 30 sets an appropriate flag (e.g., a green “proceed” flag, a yellow “caution” flag or a red “stop” flag) next to each medication order in EMR system 20.

In the preferred form of the invention, CPU 30 is configured to update the flags in EMR system 20 whenever there is a change in the patient's medical condition recorded in EMR system 20.

And in one preferred form of the invention, CPU 30 is configured to update the flags in EMR system 20 whenever there is an update in database 15 and/or rules engine 25.

It should be appreciated that CPU 30 essentially re-confirms the appropriateness of a scheduled medication delivery for the medical staff, based on the patient's current clinical information (i.e., vital signs, tests, diagnoses and other physiologic measures), just before the prescription is to be administered to the patient.

### Use of the Novel System

In general, system 5 automatically and continuously checks the appropriateness of a scheduled prescription. This is done at the time that the prescription is made, and whenever there is a change in the patient's medical condition. Thus, if a patient's medical condition changes between (i) the time that the prescription is ordered by the doctor and reviewed by a pharmacist, and (ii) the time of the administration of the medication, and/or if a patient's medical condition changes between subsequent doses of the medication, and if this change in medical condition affects the appropriateness of administration of the medication, system 5 will alert the medical staff (e.g., a nurse) prior to the medical staff administering the medication.

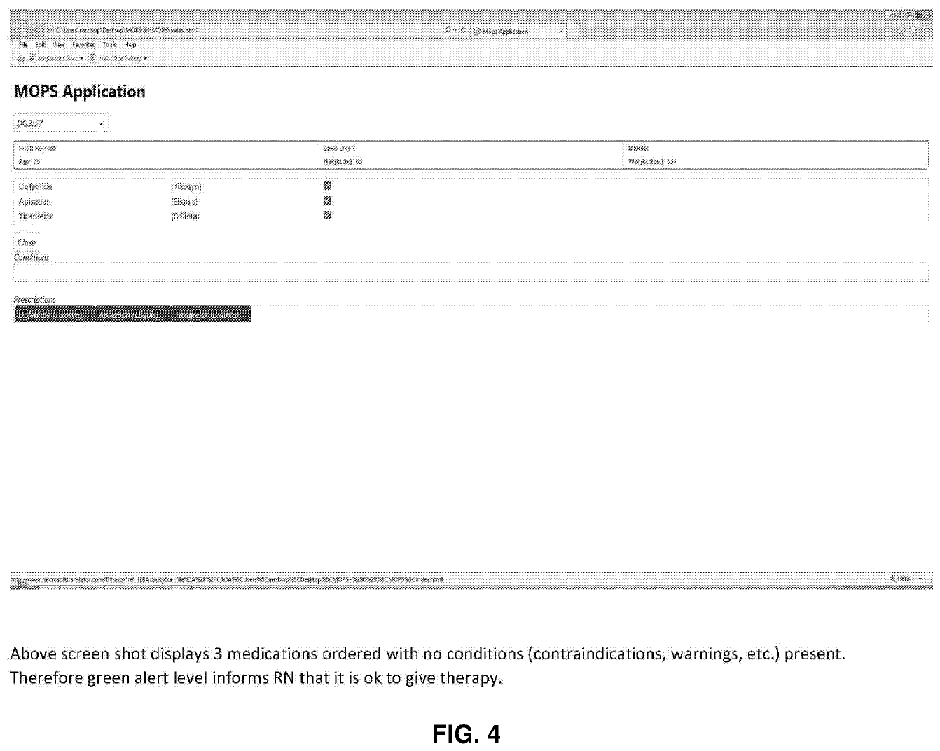

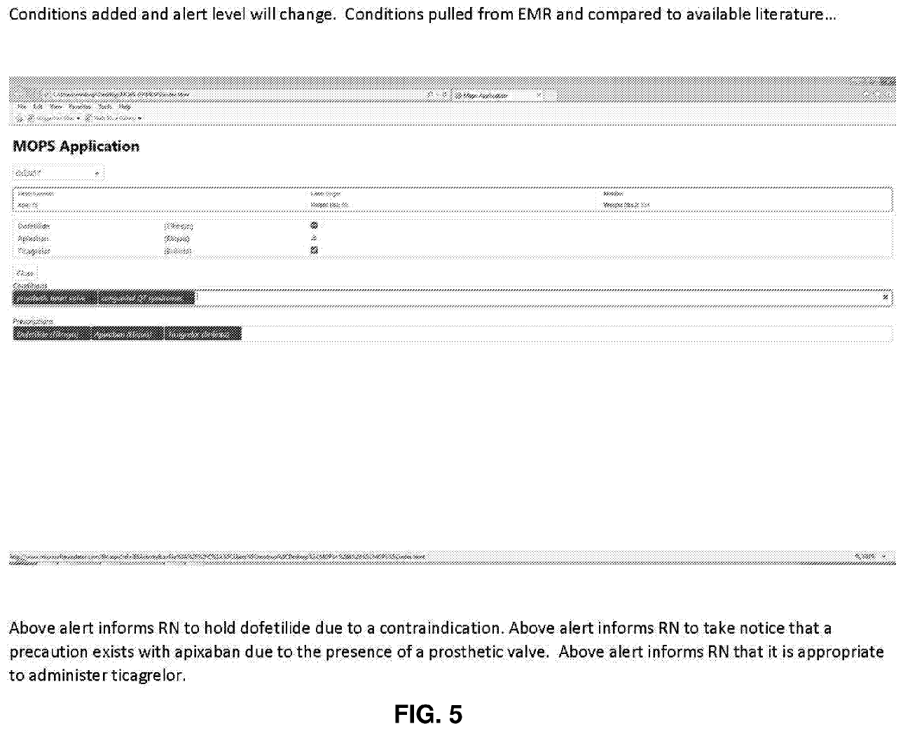

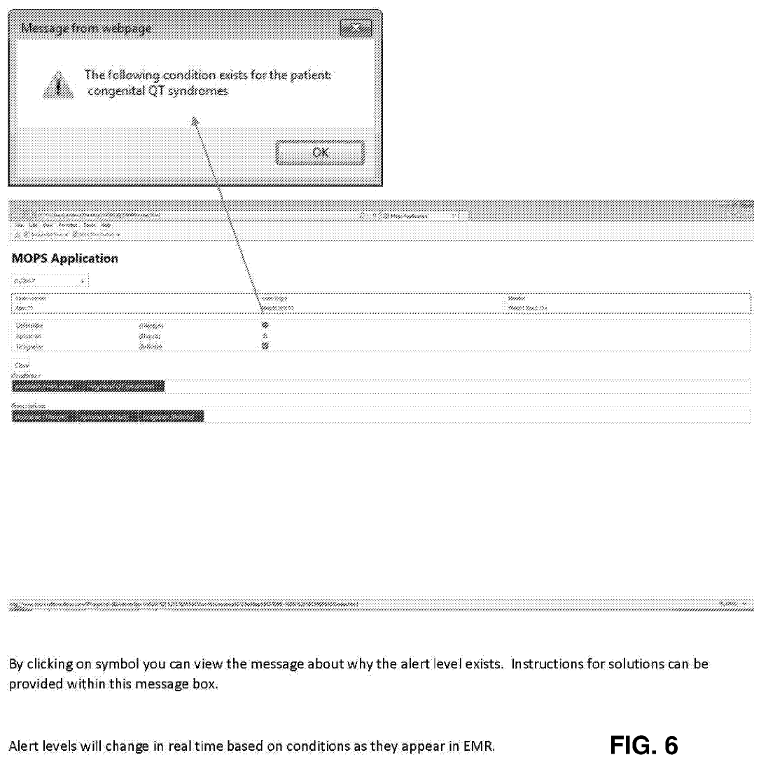

More particularly, CPU 30 receives a prescription order and determines the appropriateness of the prescription order for the patient's current medical status. This is done by CPU 30 retrieving the medication contraindications, and/or the medication warnings and precautions, for the prescribed drug from database 15, retrieving the patient's current medical status from EMR system 20, and using rules engine 25 to assess the appropriateness of the prescription order vis-à-vis the patient's current medical status. CPU 30 then assigns an appropriate flag (e.g., a green “proceed” flag, a yellow “caution” flag or a red “stop” flag) next to each medication order in the patient's medical record stored on EMR system 20. See FIGS. 4 and 5. If CPU 30 assigns a yellow “caution” flag (i.e., for when medication warning(s) and precaution(s) are indicated) or a red “stop” flag (i.e., for when medication contraindication(s) are indicated) next to a medication order, CPU 30 preferably also provides explanatory information along with the yellow “caution” flag and the red “stop” flag to explain the reason(s) for the assigned flag and any information that needs to be communicated to the prescriber of the medication or the pharmacist. See FIG. 6.

Subsequently, a member of the medical staff accesses EMR system 20 in order to check which medications need to be administered to a patient. The clinical status of the patient (e.g., physiologic measures such as weight, body temperature, heart rate, blood pressure, etc.; test results; diagnoses; progress notes; and/or other clinical information associated with a patient's medical condition) is retrieved from EMR system 20 and processed by CPU 30. CPU 30 once again compares the medication contraindications, and/or the medication warnings and precautions, stored in database 15 for each medication which is to be administered to the patient against the “real-time” clinical status of the patient from EMR system 20 and assigns a green “proceed” flag (i.e., for when no medication warning(s) and precaution(s) are indicated, and when no medication contraindication(s) are indicated), a yellow “caution” flag (i.e., for when medication warning(s) and precaution(s) are indicated) or a red “stop” flag (i.e., for when medication contraindication(s) are indicated) next to each medication order. See FIGS. 4 and 5. If a yellow “caution” flag (i.e., for when medication warning(s) and precaution(s) are indicated) or a red “stop” flag (i.e., for when medication contraindication(s) are indicated) is assigned next to a medication order, CPU 30 preferably also provides an explanation of the flag and an explanation of what needs to be communicated to the prescriber of the medication or the pharmacist. The assigned flag and explanation(s) (if necessary) are then communicated to EMR system 20 for display to the medical staff. See FIG. 6. The medical staff then sees the flags and explanation(s) (if necessary) next to the medication orders on EMR system 20 and administers/holds the medications as appropriate.

It should be appreciated that providing instructions to the medical staff in this way removes the burden of intervention decisions from the medical staff.

It should also be appreciated that automatic monitoring of a patient's medical condition reduces the risk of medication errors as a result of the time lapse between (i) when a doctor prescribes a medication and its review by a pharmacist, and (ii) when the medication is administered; and/or the time lapse between subsequent doses of a medication.

### Exemplary Architectures for Integrating an EMR System into the Automated Medication Monitoring System

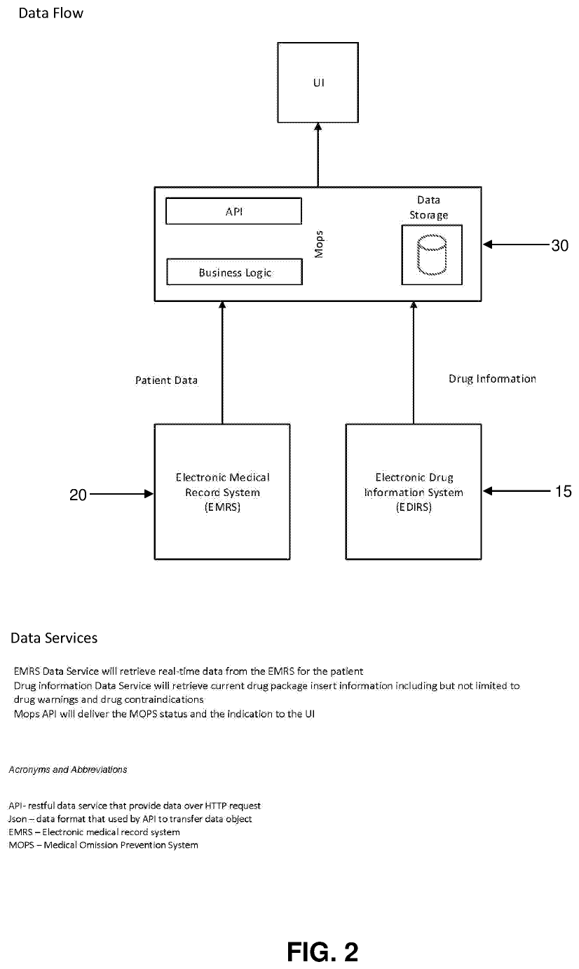

As discussed above, and as seen in FIGS. 1 and 2, novel system 5 generally comprises (i) prescription input 10, (ii) database 15, (iii) electronic medical records (EMR) system 20, (iv) rules engine 25, and (v) central processing unit (CPU) 30 (sometimes also referred to herein as “the MOPS software” and/or “the MAPS software”). And, as discussed above, EMR system 20 may comprise a commercially-available EMR system (e.g., Epic™, Cerner™, Allscripts™, etc.) or a “custom” EMR system. It will be appreciated that various architectures may be used to integrate EMR system 20 into novel system 5.

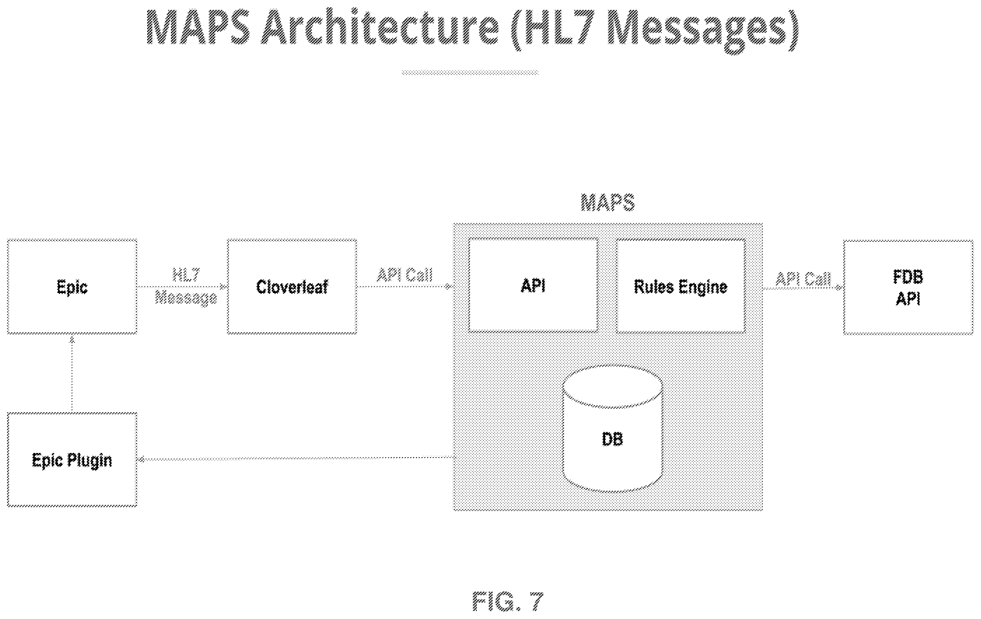

By way of example but not limitation, and looking now at FIG. 7, an EMR system 20 (e.g., the Epic EMR) may be integrated using HL7 messages.

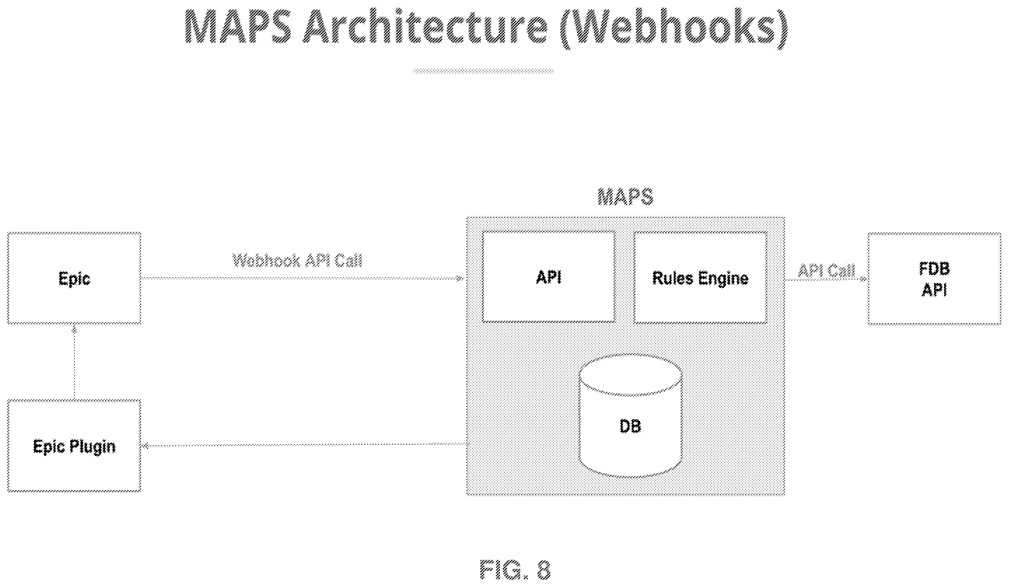

By way of further example but not limitation, and looking now at FIG. 8, an EMR system 20 (e.g., the Epic EMR) may be integrated using “webhooks”.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A system for automatically monitoring the delivery of medication to a patient, the system comprising:
an electronic medical record containing information regarding the medical condition of said patient;
a prescription order identifying a medication to be delivered to said patient;
a database identifying contraindications and warnings for specific medications;
a rules engine for determining the appropriateness of a medication vis-à-vis a medical condition of a patient; and
a central processing unit (CPU) configured to (i) provide said prescription order and said electronic medical record of said patient to said rules engine, (ii) receive a determination from said rules engine regarding the appropriateness of said prescription order vis-à-vis said medical condition of said patient, and (iii) mark said electronic medical record with an indication regarding the appropriateness of said prescription order vis-à-vis said medical condition of said patient.

2. A system according to claim 1 wherein said indication marked in said electronic medical record comprises at least one from the group consisting of a “proceed” indication, a “caution” indication and a “stop” indication.

3. A system according to claim 2 wherein, when said indication marked in said electronic medical record comprises at least one of a “caution” indication and a “stop” indication, said system also provides explanatory information.

4. A system according to claim 1 wherein said central processing unit updates its function when there is a change in said electronic medical record.

5. A system according to claim 1 wherein said central processing unit updates its function when there is a change in at least one of said electronic medical record, said database and said rules engine.

6. A system according to claim 1 wherein said indication marked in said electronic medical record comprises a flag.

7. A system according to claim 6 wherein said flag comprises a visual indicator.

8. A system according to claim 7 wherein said visual indicator comprise at least one color selected from the group consisting of green, yellow, and red.

9. A system according to claim 7 wherein said visual indicator comprises at least one shape selected from the group consisting of a box, a triangle, and a circle.

10. A system according to claim 6 wherein said flag comprises an audible indicator.

11. A system according to claim 1 wherein said prescription order further comprises at least one of a dosage and a delivery schedule.

12. A system according to claim 1 wherein said database comprises at least one of a structured database and an unstructured database.

13. A method for automatically monitoring the delivery of medication to a patient, the method comprising:
providing a system for automatically monitoring the delivery of medication to a patient, the system comprising:
an electronic medical record containing information regarding the medical condition of said patient;
a prescription order identifying a medication to be delivered to said patient;
a database identifying contraindications and warnings for specific medications;
a rules engine for determining the appropriateness of a medication vis-à-vis a medical condition of a patient; and
a central processing unit (CPU) configured to (i) provide said prescription order and said electronic medical record of said patient to said rules engine, (ii) receive a determination from said rules engine regarding the appropriateness of said prescription order vis-à-vis said medical condition of said patient, and (iii) mark said electronic medical record with an indication regarding the appropriateness of said prescription order vis-à-vis said medical condition of said patient; and
operating said central processing unit so as to (i) provide said prescription order and said electronic medical record of said patient to said rules engine, (ii) receive a determination from said rules engine regarding the appropriateness of said prescription order vis-à-vis said medical condition of said patient, and (iii) mark said electronic medical record with an indication regarding the appropriateness of said prescription order vis-à-vis said medical condition of said patient.

14. A method according to claim 13 wherein said indication marked in said electronic medical record comprises at least one from the group consisting of a “proceed” indication, a “caution” indication and a “stop” indication.

15. A method according to claim 14 wherein, when said indication marked in said electronic medical record comprises at least one of a “caution” indication and a “stop” indication, said system also provides explanatory information.

16. A method according to claim 13 wherein said central processing unit updates its function when there is a change in said electronic medical record.

17. A method according to claim 13 wherein said central processing unit updates its function when there is a change in at least one of said electronic medical record, said database and said rules engine.

18. A method according to claim 13 wherein said indication marked in said electronic medical record comprises a flag.

19. A method according to claim 18 wherein said flag comprises a visual indicator.

20. A method according to claim 19 wherein said visual indicator comprise at least one color selected from the group consisting of green, yellow, and red.

21. A method according to claim 19 wherein said visual indicator comprises at least one shape selected from the group consisting of a box, a triangle, and a circle.

22. A method according to claim 18 wherein said flag comprises an audible indicator.

23. A method according to claim 13 wherein said prescription order further comprises at least one of a dosage and a delivery schedule.

24. A method according to claim 13 wherein said database comprises at least one of a structured database and an unstructured database.
